(19) United States
(12) Patent Application Publication
(10) Pub. No.: US 2024/0395376 A1
(43) Pub. Date: Nov. 28, 2024

(54) RESEARCH DATA DISCOVERY SYSTEM AND METHOD

(71) Applicant: ROSWELL PARK CANCER INSTITUTE CORPORATION HEAL RESEARCH, INC., Buffalo, NY (US)

(72) Inventors: Mohammad K. ZIA, Buffalo, NY (US); Kevin H. ENG, Buffalo, NY (US); Chrstopher J. DARLAK, Buffalo, NY (US); Ben PLESSINGER, Buffalo, NY (US); Carl MORRISON, Buffalo, NY (US)

(21) Appl. No.: 18/689,329

(22) PCT Filed: Sep. 8, 2022
(86) PCT No.: PCT/US22/42867

§ 371 Date: Mar. 5, 2024

Related U.S. Application Data
(60) Provisional application No. 63/241,692, filed on Sep. 8, 2021.

(51) Int. Cl. G16H 10/60; G16H 10/20; G16H 50/70

(52) U.S. Cl. CPC G16H 10/60; G16H 10/20; G16H 50/70

(57) ABSTRACT

A system and method to extract data from disparate sources and connect them in a meaningful yet de-identified way allows researchers to explore the connected data and build cohorts. There are three parts to the description-first, a description of the data types and their sources and the extraction and transformation process. Second, an overview of the technologies that underpin RDDS. Third, a description of the web-portal that facilitates searching tumors and building cohorts.

### CROSS REFERENCE TO RELATED APPLICATIONS

This application claims priority benefit to U.S. Provisional Patent Application Ser. No. 63/241,692, filed Sep. 8, 2021, pending, which is hereby incorporated by this reference in its entirety as if fully set forth herein.

### BACKGROUND OF THE INVENTION

Currently, researchers laboriously sift through disparate clinical and research data to build cohorts to investigate their research ideas. Sometimes researchers do not have access to all the available data because it is inaccessible or unknown, hindering them from building robust cohorts. To address this issue, Roswell IT has developed a tool that can extract data from disparate sources and connect them in a meaningful yet de-identified way, allowing researchers to quickly build cohorts to determine the feasibility of their research ideas.

### SUMMARY OF THE INVENTION

In accordance with the purpose(s) of this invention, as embodied and broadly described herein, this invention, in one aspect, relates to a method of providing clinical and research data of patients from disparate data sources, includes extracting patient records from a plurality of disparate data sources native format, wherein each patient record comprises an associated medical record number and a valid date stamp; transforming the extracted patient records into relative event timepoints using an anchor date; linking related ones of the relative timepoints using the associated medical record numbers; providing a patient identification number to the linked relative event timepoints; storing the linked relative event timepoints as non-protected health information in a database.

In another aspect, the invention relates to a system for providing searchable access to de-identified patient data based on a timeline of medical events for the purposes of research includes a first server comprising a front-end framework, a back-end framework, a search engine, an analytic engine, a database/container; and a second server in electronic communication with the first server and in electronic communication with at least one medical data source, the second server comprising a processor, the processor comprising instructions which, when executed by processor, cause the processor comprising: extracting patient records from the at least one medical data source in native format, wherein each patient record comprises an associated medical record number and a valid date stamp; transforming the extracted patient records into relative event timepoints using an anchor date; linking related ones of the relative timepoints using the associated medical record numbers; providing a patient identification number to the linked relative event timepoints; and communicating the linked relative event timepoints as non-protected health information (non-PHI) with an anonymized patient identification number to the first server storing thereon the non-PHI, whereby the non-PHI is anonymized data.

In yet another aspect, the invention relates to a web portal includes a graphical user interface comprising a display with a workspace; a search interface hosted in the graphical user interface, the search interface providing to a user a graphical representation of search queries to be implemented by a search engine connected to the search interface and in electronic communication with at least one communications portal for sending selected search queries to a database hosted on a server comprising a database of non-protected health information stored as a collection of event timepoints of health events for a given patient.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIG. 1 shows the structure of a system according to principles described herein and how users and administrators interact with it.

FIG. 2 shows the search user interface according to principles described herein.

FIGS. 3A-3C illustrate summary statistics displayed by system when a user hits search.

FIG. 4 illustrates a sample search results displayed as a timeline.

FIG. 5 shows the effect of hovering a pointing device over one of the events. Here an administered drugs event is shown.

FIG. 6 shows the options available for rescaling the timeline.

FIG. 7 shows options for focusing and panning the timeline.

FIG. 8 illustrates how researchers can sort the timeline based on patientid, age of diagnosis (AgeDx), sex, patient status (Alive/Dead), and Survival

FIG. 9 illustrates how researchers can filter, highlight or auto-select by any term available in the timeline (e.g., drug, class of drug, health issue, type of radiology scan, etc.).

FIG. 10 illustrates how researchers can save patients/tumors of interest by clicking on the checkbox next to the patientid-seqprim and clicking “save selected.” Researchres have the option to save to a new group or to an existing group.

FIG. 11 illustrates when a researcher saves patients/tumors to a group, these patients/tumors become accessible in their respective tumor tab. FIG. 11 shows four groups and the count of patients/tumors next to the group name.

FIG. 12 illustrates how Inside the “All” tab, each patientid-seqprim is followed by a filled circle whose color matches group tab color. This is to enable researchers to quickly know which group a patient/tumor belongs to.

FIG. 13 illustrates how, in the output tab, Researchers can view the characteristics table, contains age, primary site, histology, grade, and stage breakdown by group and overall. Researchers can also download the table, patient information, and the events for further analysis.

FIG. 14 illustrates how Researchers can view the Kaplan-Meier estimator curve in the output tab.

FIG. 15 illustrates an example system architecture and operation of a system architecture according to principles described herein.

### DESCRIPTION OF AN ILLUSTRATIVE EMBODIMENT

The present invention may be understood more readily by reference to the following detailed description of preferred embodiments of the invention and the Examples included therein and to the Figures and their previous and following description.

It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Described herein is a web-based tool that connects clinical and research data of patients from disparate data sources to produce de-identified time events relative to the date of cancer diagnosis. While described herein with respect to cancer and “tumors,” other medical conditions, signs and symptoms can be resourced and identified using the principles described herein. In other words, the data and data fields in the databases accessed can be related to information other than cancer, but cancer is used herein as an example.

As described herein, the relative sequence of time events can be compared and contrasted across 100s of patients by researchers to build cohorts for grants, studies, and publications. Currently, the tool connects the following data types: Disease (diagnostic, recurrence information, and patient status), Intervention (prescribed and administered drugs and surgical procedures), Biospecimen (solid tumor, liquid, and tissue microarray), Diagnostic (clinical genomic and single analyte), and Research Data (research sequencing and epidemiological questionnaires). In addition, the tool generates statistical outputs such as disease characteristics summary and Kaplan Meier curves to guide the researchers to build meaningful cohorts. It can also export de-identified patient-level and event-level data as a delimited text file for further analysis using external tools. In the future, the tool will be enhanced to include more data sources such as radiation medicine treatments, radiographic evaluations, pathology results, general lab (focused result sets), and genomic mutational searching (clinical and research). It will also support choosing different reference events to calculate the relative time of all other events. In addition, it will allow filtering of patients based on the desired sequence of events.

Reference will now be made in detail to the present preferred embodiment(s) of the invention, an example(s) of which is [are] illustrated in the accompanying drawings. Wherever possible, the same reference numbers are used throughout the drawings to refer to the same or like parts.

Although various data sources are described herein, the data discovery system described herein can connect limitless data sources. However, the present illustrated embodiment relates to cancer data, and makes reference to Cancer Registry as a data source because it provides the date of diagnosis of cancer, which is used as the reference even to calculate the relative time of all other events.

Table 1 shows the current list of data types and sources and the fields extracted from them along with a brief description where necessary. Note, in this embodiment, a medical record number (MRN) is used across all data types to connect data sources (or link data derived therefrom). That said, MRN is a currently used linking mechanism, but other linking mechanisms are possible. Other data types or sources are possible, and thus not limited to those described herein. Fields associated with other data sources are possible.

TABLE 1

| Data Type/Data Source | Fields |
|---|---|
| Data Type: Disease and | MRN |
| Recurrence Information | TumorID |
| Data Source: Cancer | Tumor Description |
| Registry | SeqPrim (number to indicate instances of |
| | cancer for a given patient) |
| | Date of Diagnosis |
| | Date of Admission (used in case date of |
| | diagnosis is missing as an approximation |
| | of the date of diagnosis) |
| | Primary Site code and description |
| | Histology code and description |
| | Tumor Grade (pre-2018) |
| | Tumor Clinical Grade (post-2018) |
| | Tumor Pathological Grade (post-2018) |
| | Tumor Grade Post Therapy (post-2018) |
| | Laterality Description |
| | Tumor, node and metastasis (TNM) edition |
| | Tumor Clinical Stage Group |
| | Tumor Pathology Stage Group |
| | Tumor PostRx Stage Group |
| | Recurrence Date (if present) |
| | Recurrent Description |
| Data Type: | MRN |
| Intervention - | Drug Name |
| Prescribed Drugs | Date prescribed |
| Data Source: EHR | Route |
| | Prescription instructions |
| | Prescribing entity (Internal/External) |
| Data Type: | MRN |
| Intervention - | Drug Name |
| Administered Drugs | Therapeutic Category (contains both parent |
| Data Source: EHR | and child category) |
| | Date performed |
| | Summary of event |
| | Dose |
| | Dose unit measure |
| | Route |
| Data Type: | MRN |
| Intervention - | Procedure Description |
| Surgical Procedures | |
| Data Source: PICIS, | |
| LIMS, and Cancer | |
| Registry | |
| Data Type: | MRN |
| Biospecimen - | Sample ID |
| Solid Tumor | Date of procurement |
| (Frozen Tissue) | Tissue Description |
| Data Source: LIMS | Disease Description |
| | PMR |
| | Quantity available |
| Data Type: | MRN |
| Biospecimen - | Collection Date |
| Liquid (DBBR | Collection Type |
| collections) | |
| Data Source: LIMS | |
| Data Type: | MRN |
| Biospecimen - TMAs | TMA Description |
| Data Source: LIMS | TMA Date |
| Data Type: | MRN |
| Diagnostic - | Date |
| Clinical Genomic | Test Company |
| Data Source: | Test Type |
| Custom non-vendor | |
| database that | |
| stores proprietary | |
| reports | |
| Data Type: | MRN |
| Diagnostic - | Date |
| Single Analyte | Test Type |
| Data Source: | |
| Custom non-vendor | |
| database that | |
| stores proprietary | |
| reports | |
| Data Type: | MRN |
| Research Data - | Sequencing Type |
| Research Sequencing | Sequencing Library |
| Data Source: Custom | Date |
| non-vendor database | |
| that stores | |
| proprietary reports | |
| Data Type: Research | MRN |
| Data - Epidemiological | Date |
| questionnaires | |
| Data Source: LIMS | |
| Data Type: | MRN |
| Demographics | Patient ID (a deidentified unique identifier of |
| Data Source: | patients) |
| Patient Master and | Date of Birth |
| Cancer Registry | Date of Death |
| | Date of Last Contact |
| | Patient Status (Alive/Dead) |
| | Sex |
| | Race(s) |
| | Hispanic Status |
| | Alcohol Usage |
| | Tobacco Usage |

### Data Extraction, Transformation, and Loading

For each data type, data is extracted from its respective source using, for example, a Python script and stored in a database, e.g., an SQLite3 database. Next, various transformations are performed to prepare the data. For Cancer Registry, all tumor grade descriptions are mapped from coded fields to site specific descriptions. For administered drugs and prescribed drugs, brand names may be mapped to generic names to make searching more consistent. For demographics, discrepancies between patient master and cancer registry are reconciled.

At this point the transformed data sources are linked using the MRN (or other identifier) to produce a tumor-centric output, i.e., all information that can be associated with a tumor is collated. Therefore, for a given row from any data source, its relative days, months, years, natural log of months, and percent (0% is the date of diagnosis—100% is the date of last contact or date of death) from the date of diagnosis is calculated, transforming the row into a tumor event relative to the date of cancer diagnosis. For each tumor event type, an appropriate display label is also generated. Table 2 lists the label/label content for each tumor event type. Note, that while this embodiment illustration is made with respect to cancer tumor, the present systems and methods can be used to link and study other relevant medical/diagnostic data.

TABLE 2

| Tumor Event Type | Label Content |
|---|---|
| Diagnosis | Age at diagnosis, sex, primary site description, |
| | histology, laterality description, grade |
| | description (pre-2018) or grade clinical, |
| | pathological, and post therapy (post-2018) |
| | description, clinical stage group description, |
| | pathological stage group description, postrx |
| | stage group description, and TNM edition. |
| First Recurrence | Recurrence description |
| Disease/Patient | Death Status or Last contact by cancer registry |
| Status | |
| Prescribed Drugs | generic drug name and parent and child |
| | therapeutic categories |
| Administered Drugs | generic drug name and parent and child |
| | therapeutic categories |
| Surgery | Surgery description |
| Frozen Tissue | Tissue description, PMR, and quantity |
| DBBR Collections | None |
| TMAs | TMA name and description |
| Clinical Genomics | Performing company and test type |
| Single Analyte Test | Performing company and test type |
| Sequencing | Sequencing type and library |
| DBBR Questionnaires | None |

Besides the tumor events, many other data fields are associated with a tumor to support multifaceted searching and generate outputs. Table 3 lists all the data fields that can be associated with a tumor, if they are directly from data source or transformed/computed, if they are searchable and how they are used. The field names correspond to Elasticsearch field names.

TABLE 3

| | Source | | |
|---|---|---|---|
| Field | Field | Searchable | Description |
| ajccstagegroupclin | Yes | No | Used in characteristics table output |
| ajccstagegrouppath | Yes | No | Used in characteristics table output |
| ajccstagegrouppostrx | Yes | No | Used in characteristics table output |
| alcoholdescription | Yes | Yes | Allows searching for patient by alcohol status |
| anatomical_group | No | Yes | This field groups primary sites based on main anatomical groups. |
| dxage | No | Yes | Calculated from the date of birth and date of diagnosis as a number with two decimal places. |
| eadmin | No | No | Contains the administered drugs events. This field has the label concatenated with days, months, |
| | | | years, log months, and percent elapsed since the date of diagnosis and filter terms (generic name, |
| | | | parent and child therapeutic category, separated by the pipecharacter). Concatenation is done |
| | | | using the group separator character. |
| ecollections | No | No | Contains the DBBR collections events. This field has the label concatenated with days, months, |
| | | | years, log months, and percent elapsed since the date of diagnosis. Concatenation is done |
| | | | using the group separator character. |
| edisease | No | No | Contains the patient status events (death or last contact). This field has the label concatenated |
| | | | with days, months, years, log months, and percent elapsed since the date of diagnosis. |
| | | | Concatenation is done using the group separator character. |
| edx | No | No | Contains the diagnostic event. This field has the label concatenated with days, months, years, |
| | | | log months, and percent elapsed since the date of diagnosis. Concatenation is done using the |
| | | | group separator character. |
| egenomic | No | No | Contains the clinical genomics events. This field has the label concatenated with days, months, |
| | | | years, log months, and percent elapsed since the date of diagnosis. Concatenation is done using |
| | | | the group separator character. |
| egsr | No | No | Contains the sequencing events. This field has the label concatenated with days, months, years, |
| | | | log months, and percent elapsed since the date of diagnosis. Concatenation is done using the |
| | | | group separator character. |
| equestionnaire | No | No | Contains the DBBR questionnaire events. This field has the label concatenated with days, |
| | | | months, years, log months, and percent elapsed since the date of diagnosis. Concatenation is |
| | | | done using the group separator character. |
| erecurrence | No | No | Contains recurrence event. This field has the label concatenated with days, months, years, log |
| | | | months, and percent elapsed since the date of diagnosis. Concatenation is done using the group |
| | | | separator character. |
| erx | No | No | Contains the prescribed drugs events. This field has the label concatenated with days, months, |
| | | | years, log months, and percent elapsed since the date of diagnosis and filter terms (generic |
| | | | name, parent and child therapeutic category, separated by the pipe character). Concatenation |
| | | | is done using the group separator character. |
| esat | No | No | Contains the single analyte events. This field has the label concatenated with days, months, |
| | | | years, log months, and percent elapsed since the date of diagnosis. Concatenation is done |
| | | | using the group separator character. |
| esurgery | No | No | Contains the surgical events. This field has the label concatenated with days, months, years, |
| | | | log months, and percent elapsed since the date of diagnosis. Concatenation is done using the |
| | | | group separator character. |
| ethnicities | No | No | Contains all the ethnic statuses associated with a patient. |
| etissue | No | No | Contains the frozen tissue collection events. This field has the label concatenated with days, |
| | | | months, years, log months, and percent elapsed since the date of diagnosis and filter terms |
| | | | (tissue path, disease path, and PMR, concatenated by the pipe character). Concatenation is |
| | | | done using the group separator character. |
| etma | No | No | Contains the TMA events. This field has the label concatenated with days, months, years, log |
| | | | months, and percent elapsed since the date of diagnosis. Concatenation is done using the group |
| | | | separator character. |
| generic_drugs | No | Yes | Contains all the generic drugs prescribed or administered to a patient. |
| gradeclinical | Yes | No | Posst-2018 grade clinical label. It is used in the characteristics output table. |
| gradedescription | Yes | No | Pre-2018 grade description. It is used in the characteristics output table. |
| gradepathological | Yes | No | Post-2018 grade pathological description. It is used in the characteristics output table. |
| gradeposttherapy | Yes | No | Post-2018 grade post therapy description. It is used in the characteristics output table. |
| has_administered_drugs | No | Yes | Field allows searching for patients that have administered drugs events. |
| has_clinical_genomics | No | Yes | Field allows searching for patients that have clinical genomic events. |
| has_collections | No | Yes | Field allows searching for patients that have DBBR collection events. |
| has_disease_status | No | Yes | Field allows searching for patients that have patient disease status events. |
| has_frozentissues | No | Yes | Field allows searching for patients that have frozen tissue collection events. |
| has_gsr | No | Yes | Field allows searching for patients that have sequencing events. |
| has_multiple_disease_status | No | Yes | Field allows searching for patients that have multiple patient disease status. |
| has_multiple_tumors | No | Yes | Field allows searching for patients that have multiple tumors. |
| has_prescribed_drugs | No | Yes | Field allows searching for patients that have prescribed drug events. |
| has_questionnaires | No | Yes | Field allows searching for patients that have DBBR questionnaire events. |
| has_recurrence | No | Yes | Field allows searching for patients that have recurrence event. |
| has_sat | No | Yes | Field allows searching for patients that have single analyte testing events. |
| has_surgeries | No | Yes | Field allows searching for patients that have surgical events. |
| has_tmas | No | Yes | Field allows searching for patients that have TMA events. |
| hispanic | Yes | Yes | Indicates a patient's Hispanic status |
| histology_combined | No | Yes | Combines histology code and description to allow searching by both. |
| histologydescription | Yes | No | Is used in the characteristics output table |
| parent_therapeutic_categories | No | Yes | Contains all the parent therapeutic categories associated with any administered or prescribed |
| | | | drugs given to a patient. |
| patientid | No | No | De-identified patient identifier |
| patientid_seqprim | No | No | De-identified patient identifier combined with SeqPrim |
| patientstatus | Yes | Yes | Dead or Alive |
| pnsr_sample_diseases | No | Yes | All the disease paths from frozen tissue samples for a patient. |
| pnsr_sample_pmr | No | Yes | All the PMR from frozen tissue samples for a patient. |
| pnsr_sample_tissues | No | Yes | All the tissue path from frozen tissue samples. |
| primary_site_combined | No | Yes | Primary site code and description combined to allow searching by both. |
| primarysitedescription | Yes | No | Is used in the characteristics table in the output. |
| ptids | No | Yes | Contains patientid and patientid_seqprim. This field allows searching a patient by either |
| | | | just their patientid or their patientid with seqprim combined. |
| races | No | Yes | All the races associated with a patient. |
| sex | No | Yes | Patient's sex. |
| survival-months | No | No | Months between the date of diagnosis and date of death or date of last contact. |
| therapeutic_categories | No | Yes | Contains all the child therapeutic categories associated with any administered or |
| | | | prescribed drugs given to a patient. |
| tma_descs | Yes | Yes | TMA labels for searching TMAs |
| tobaccodescription | Yes | Yes | Allows searching by patient's tobacco usage |
| trg | No | Yes | Some primary sites are rolled up into translation research groups. This allows searching |
| | | | primary sites based on TRG. |
| tumordeid | No | No | One-way de-identification of Cancer Registry tumor ID. Used in the backend to make links. |

To summarize, for each tumor in cancer registry, patient data from disparate sources are linked using MRN and transformed to events by calculating the relative time of the data from the date of diagnosis. At this point all PHI fields, dates and MRNs, are dropped because they are no longer needed and only non-PHI tumor data, events, and patient demographics useful for searching are stored in an intermediary SQLite3 database. Each tumor is indexed using a combination of patient id, which is deidentified unique identifier for each patient at Roswell Park and SeqPrim, which identifies an instance of patient tumor, for example PT-00295122-02. The stored data is then loaded into Elasticsearch.

Note that for complete segregation of PHI data and non-PHI data, the extraction and transformation is done on a separate server from the server that hosts the backend, frontend, and the Elasticsearch database.

### Technology Overview

The system described herein may integrate opensource technologies. Table 4 lists each exemplary open source technologies as examples, its purposes, and license. It is possible to substitute other technology/program/code, as appropriate, without departing from the spirit and scope of the invention.

TABLE 4

| # | Technology | Purpose | License |
|---|---|---|---|
| 1 | Python | Programming language used to | Python |
| | | write extraction, transformation, | Software |
| | | and loading (ETL) code. | Foundation |
| | | | License |
| 2 | Django | Web framework used for building | 3-clause |
| | | the web-portal backend. | BSD |
| 3 | Django | Integrates with Django to provide | 3-clause |
| | Restframework | REST-API features used by the | |
| | | frontend communicate with the | |
| | | backend. | |
| 4 | Simple JWT | Provides support for Web Token | MIT |
| | | Authentication for active | License |
| | | directory Authentication. | |
| 5 | ADFS | Provides support Active directory | BSD-2- |
| | Authentication | authentication for Django. | Clause |
| | for Django | | License |
| 6 | django-model- | A supporting package for Django | BSD-3- |
| | utils | for keeping track of creation and | Clause |
| | | modification dates of rows in | License |
| | | database tables. | |
| 7 | python-dotenv | Supports accessing database | BSD-3- |
| | | credentials in a safe way. | Clause |
| | | | License |
| 8 | Gunicorn | A Python Web Server Gateway | MIT |
| | | Interface HTTP server. | License |
| 9 | Pandas | Python package for data analysis. | BSD-3- |
| | | | Clause |
| | | | License |
| 10 | Lifelines | A survival analysis package for | MIT |
| | | Python used to generate Kaplan- | License |
| | | Meier curve. | |
| 11 | Elasticsearch | Main database that supports fast | Elastic |
| | | searching of tumors and events. | License |
| | | | 2.0 |
| 12 | MariaDB | Database for user authentication, | GPLv2, |
| | | workspace management, search | LGPLv2.1 |
| | | management, and logging. | (client |
| | | | libraries) |
| 13 | mysqlclient | Allows Python to connect to | GPL-2.0 |
| | | MariaDB | License |
| 14 | SQLite3 | Used for storing extracted and | Public |
| | | transformed data. | Domain |
| 15 | Angular | Frontend technology that powers the | MIT |
| | | web-portal | License |
| 16 | Angular | Provides support for website layout, | MIT |
| | Material | UI components, and themes. | License |
| 17 | PrimeNG | Provides additional UI components | MIT |
| | | | License |
| 18 | Angular | Provides multiselect UI component | MIT |
| | ng-select | | License |
| 19 | Nginx | Webserver for serving the backend | 2-clause |
| | | and frontend | BSD |
| 20 | Docker | Allows running services as self- | Apache |
| | | contained Docker containers | License |
| | | | 2.0 |

According to principles described herein two servers support the complete segregation of PHI and non-PHI.

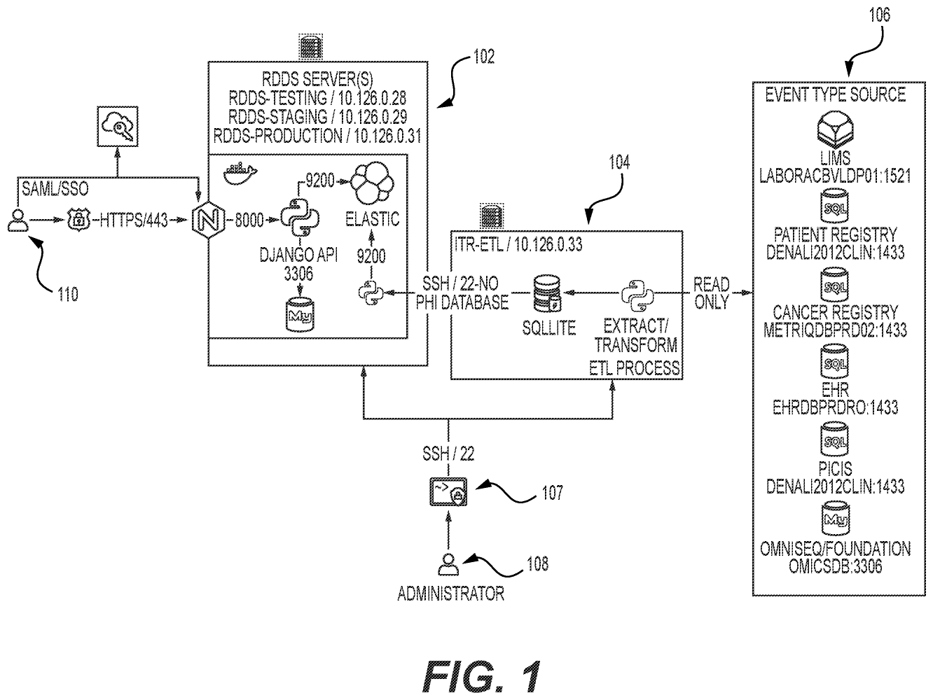

FIG. 1 shows the structure of the research data discovery system (“RDDS”) according to principles described herein and how users and administrators interact with it. FIG. 1 shows the two servers: “RDDS Servers” 102 and “ETL Process server” 104. The ETL Process server 104 is responsible for using Python scripts to automatically extract data from various sources 106, as outlined in the tables above. For example, the ELT server extracts the data from the sources 106, such as databases, transforms the data, combines the data, and saves the data in a SQLite3 database. In an aspect, this SQLite3 database is then transferred over to the RDDS server, which can load the data into an analytical engine, such as Elasticsearch. In an implementation, the RDDS server runs the frontend (Angular), backend (Django), Elasticsearch, MariaDB, and Nginx as a service inside docker containers. These containers combined provide the functionality of RDDS. For security purposes, Secure Shell (“SSH”) access 107 may be used to limit access to only administrators 108 on both the RDSS and the ETL servers. Users 110 only interact with the RDDS server, so the users 110 never interact with the ETL process serve 104 r, which contains protected health information (“PHI”) information.

### RDDS Web-Portal Functionality

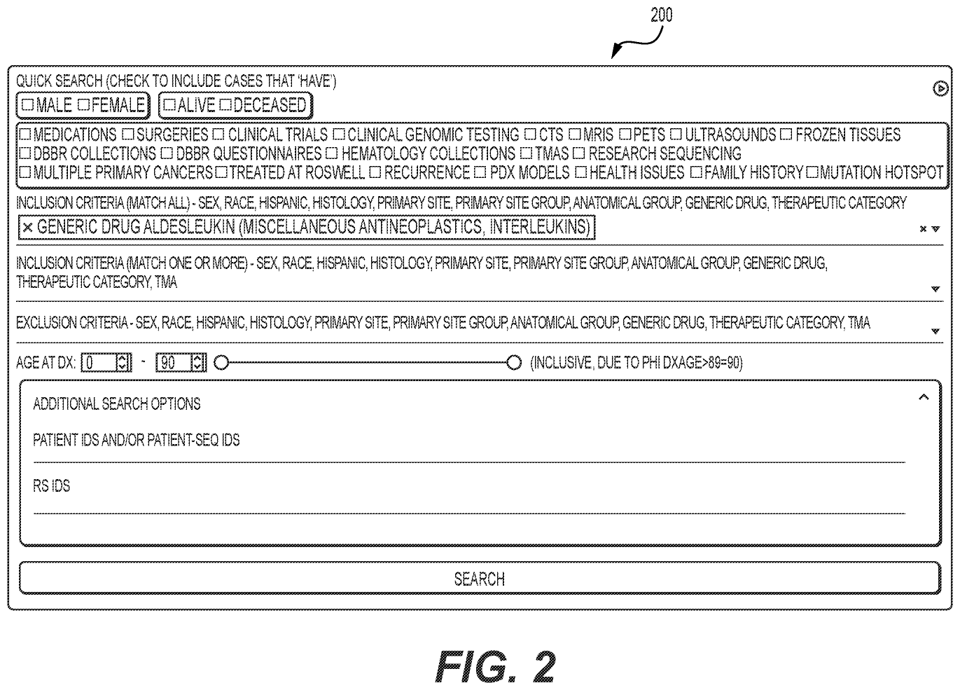

The RDDS web-portal provides the user functionality needed to build cohorts. To build a cohort, researchers begin by creating a workspace. A workspace is a container that allows researchers to perform searches and save results of interest into groups. FIG. 2 shows an example search user interface (UI) 200, which may be a graphical user interface. For example, as illustrated in FIG. 2, researchers/users can search based on a variety of criteria/parameters, e.g. a researcher may search for tumors of interest based on Sex, Race, Hispanic Status, Primary Site, Primary Site Group, Anatomical group, Generic Drug Name, Therapeutic Category (parent and child), and TMA description. These criteria can either be inclusion “match all”, meaning AND, inclusion “match one or more”, meaning OR, or exclusion, meaning NOT. In addition, researchers can search based on age of diagnosis and patient id. The search parameters defined by the user interface can vary based on the information available from the various data sources and therefore should not be limited based on current examples.

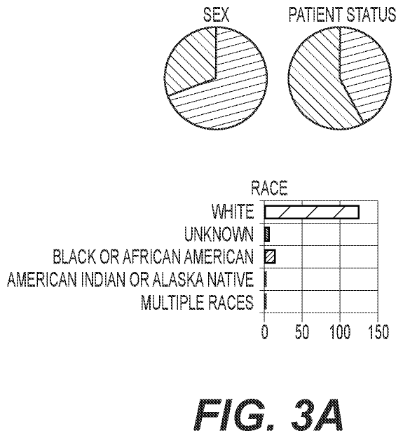

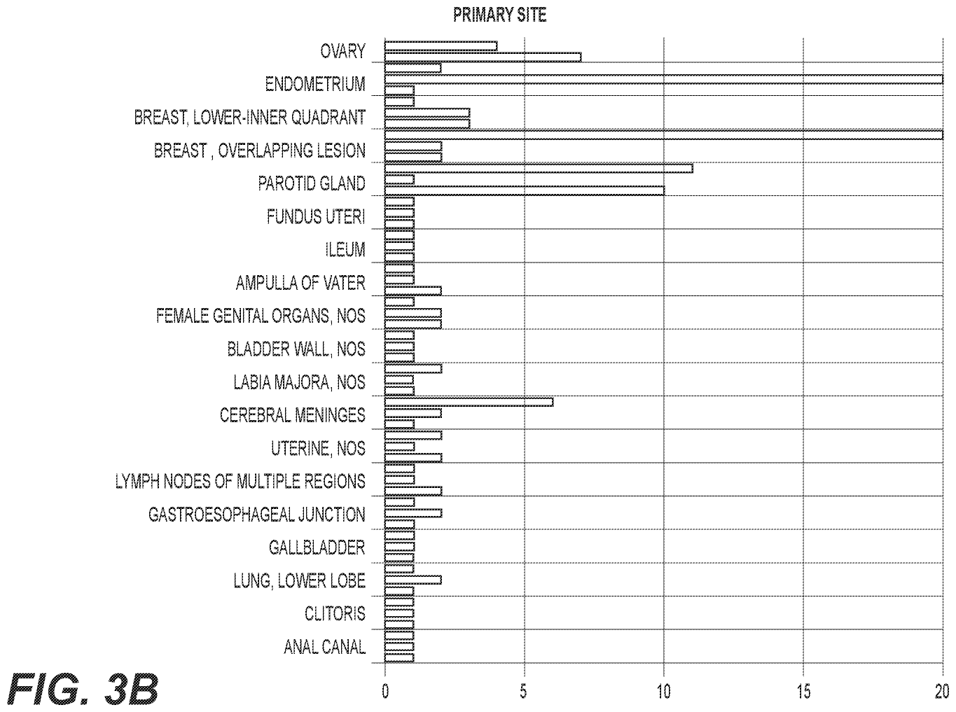

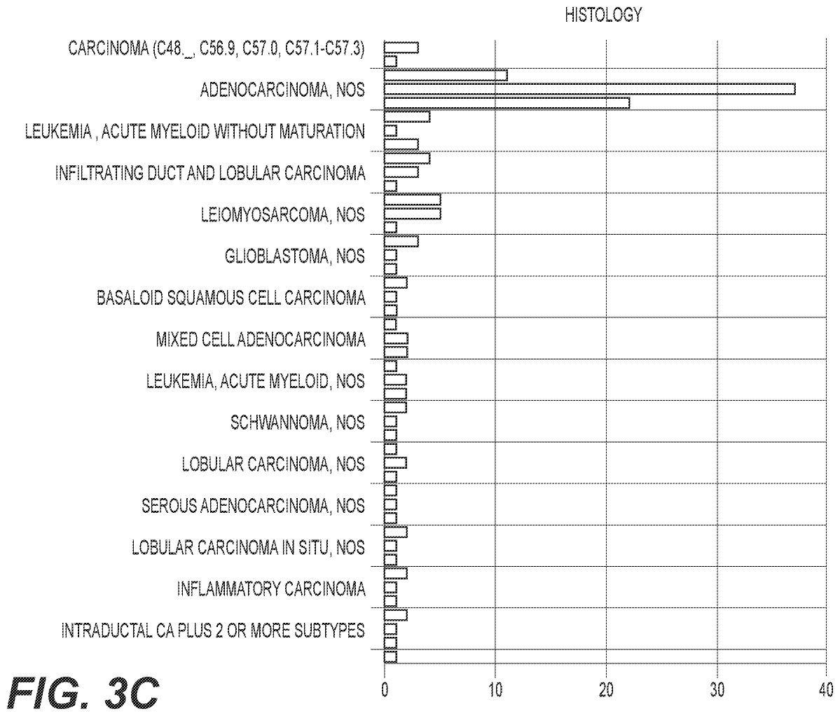

FIGS. 3A-3C show examples of summary statistics displayed by RDDS when a user hits search. For example, after a researcher hits “search”, they are first shown summary statistics of their results. examples of summary statistics are shown in FIGS. 3A-3C and, for example, may include sex (FIG. 3A), patient status (dead or alive)(FIG. 3A), race (FIG. 3A), primary site (FIG. 3B), and histology (FIG. 3C). Other summary statistics can be shown,

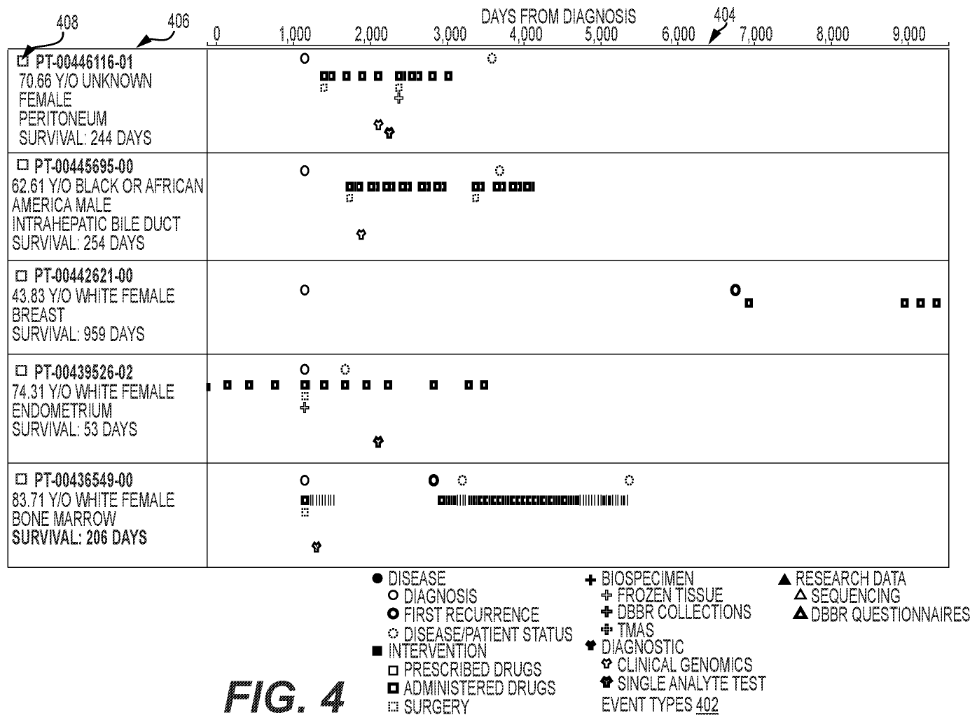

Under the summary statistics, researchers can view the timeline for patient/tumors that match their search criteria. FIG. 4 shows a sample search results displayed as a timeline, e.g. an example timeline. On the right side, the various event types 402 and their respective symbol are shown. In the center, the events 404 are displayed. On the left side, Patient ID, sex, primary site, and survival (months since diagnosis, green for alive and red for dead) 406 are shown along with a checkbox 408 to select patient/tumor of interest and save to a group.

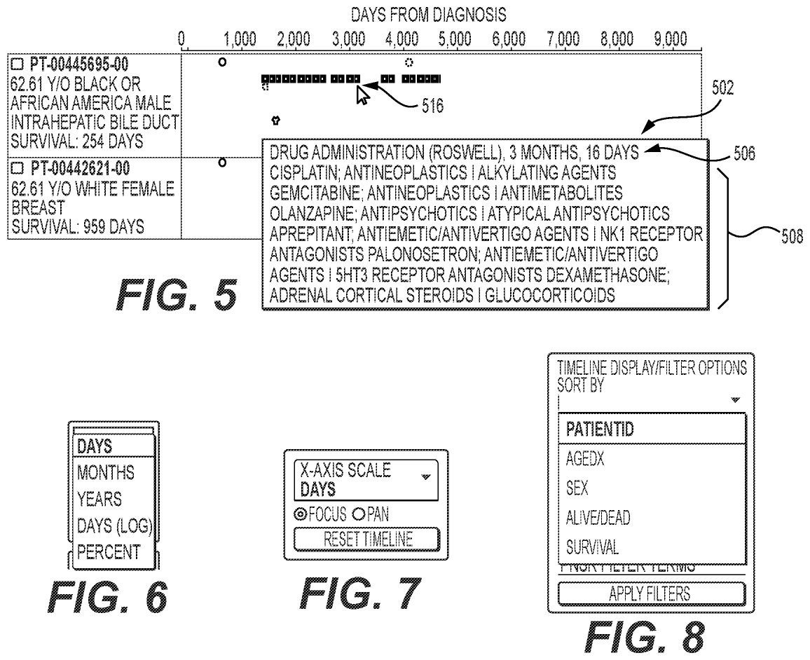

Researchers can “hover” over each event to get more information about the event. FIG. 5 shows a sample hover display 502 when a researcher hovers a pointing device, such as a mouse cursor 516, over an event identifier on the user interface screen. Once the pointing device is over the event identifier or information, such as a data point, more information is available via a pop up window 502. For example, using this method, the researcher can see the relative elapsed time 506 since the date of diagnosis and information 508 about the events, which are the labels associated with the events. See Table 2 for description of label for each event type.

FIG. 5 shows the effect of hovering over one of the events. Here an administered drugs event is shown. Researchers can control the time scale of the timeline, see FIG. 6. FIG. 6 shows the options available for rescaling the timeline. Researchers can focus (zoom) and pan the timeline, see FIG. 7. FIG. 7 shows options for focusing and panning the timeline.

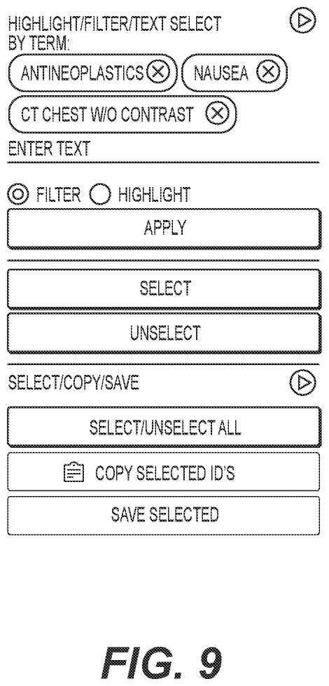

Researchers can sort the timeline by patientid, age of diagnosis (AgeDx), sex, patient status (Alive/Dead), and survival, see FIG. 8. Researchers can filter the administered and prescribed drug events that are displayed based on generic drug names, parent and child therapeutic categories. They can also filter the frozen tissue time events based on disease, tissue, and PMR. FIG. 9 shows these display options. For example, users can highlight or auto-select by any term available in the timeline (e.g. drug, class of drug, health issue, type of radiology scan etc.).

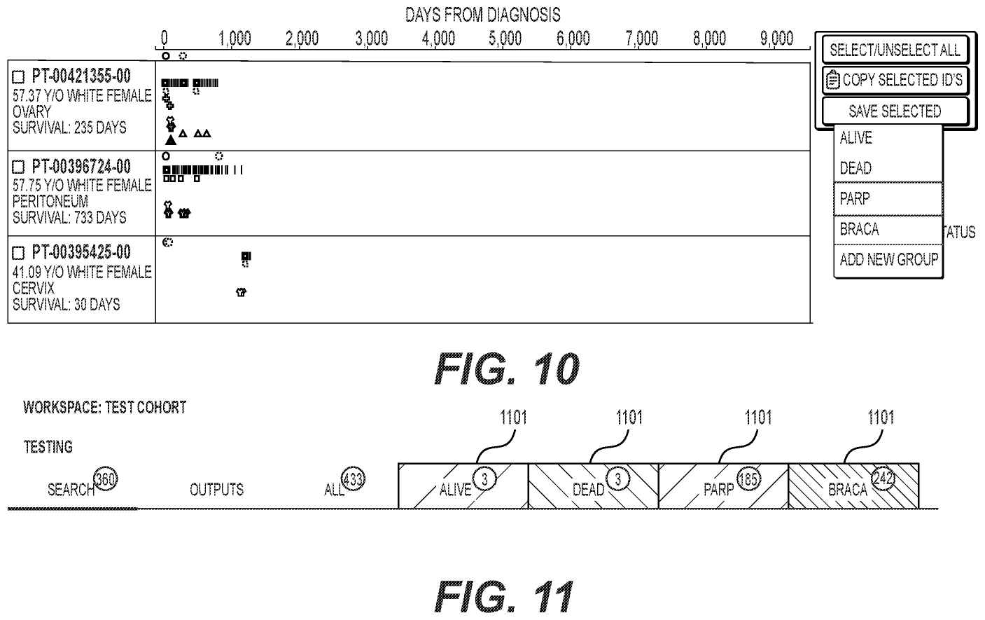

Researchers can save patient/tumors of interest to groups by checking the checkbox next to the patientid-seqprim and clicking “save selected,” see FIG. 10 for example. Researchers can save to an existing group or to a new group. As illustrated in FIG. 10, researchers can save patients/tumors of interest by clicking on the checkbox next to the patientid-seqprim and clicking “save selected.” Researchers have the option to save to a new group or to an existing group. Once a researcher has saved patients/tumors to a group, they are removed from search results and are now available in the group tab 1101 that is accessible from the top of the workspace, see FIG. 11. As illustrated in FIG. 11, when a researcher saves patients/tumors to a group, these patients/tumors become accessible in their respective tumor tab. FIG. 11 shows four groups and the count of patients/tumors next to the group name, but more or fewer groups can be created according to a researcher's needs.

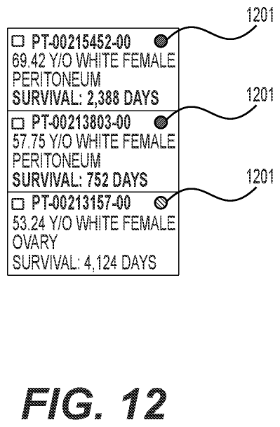

If there are more than one group, the “All” group appears that shows all the patients in a single tab along with their group-tab color as a filled circle 1201 next to the patientid-seqprime for easily distinguishing which patient/tumor belongs to which group, see FIG. 12. As illustrated in FIG. 11, inside the “All” tab, each patientid-seqprim is followed by a filled circle whose color (represented by hatching) matches group tab color (represented by hatching). This is to enable researchers to quickly know which group a patient/tumor belongs to.

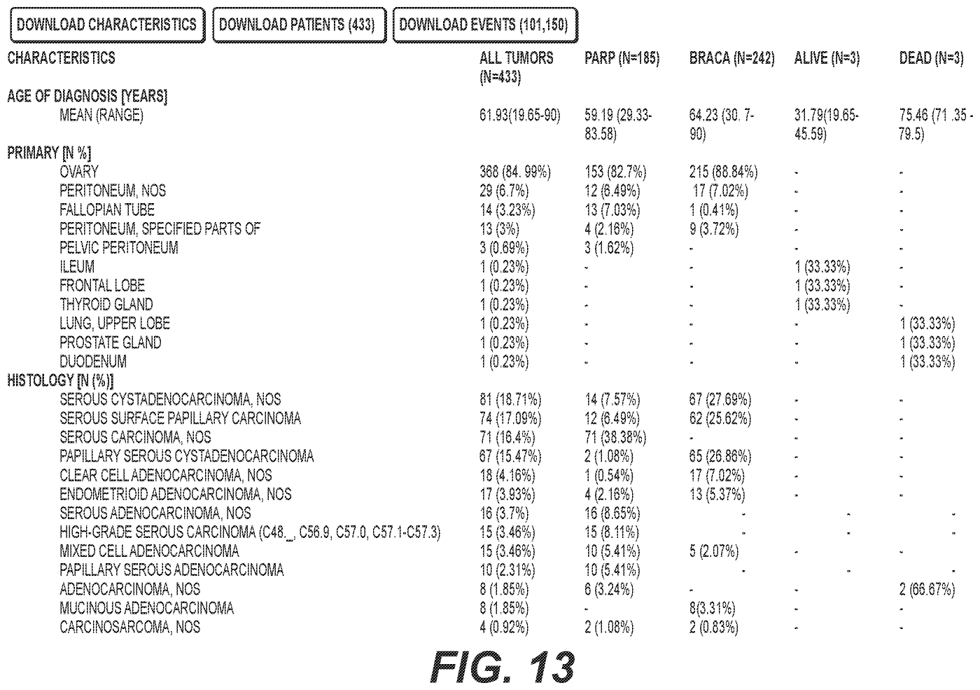

After a researcher adds patients/tumors to a group, an output tab appears at the top of the workspace (not shown). Activating this tab causes display of a characteristics table, as illustrated in FIG. 13. The characteristics table may include age, primary site, histology, grade, and stage breakdown by group and overall. Researchers can also download the table, patient details, and events, see FIG. 13. Referring to FIG. 13, in the output tab, Researchers can view the characteristics table, contains age, primary site, histology, grade, and stage breakdown by group and overall. Researchers can also download the table, patient information, and the events for further analysis.

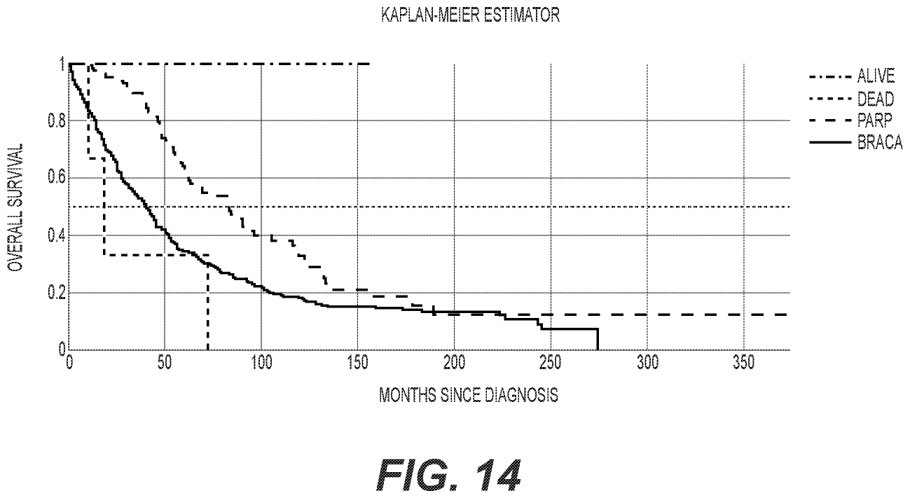

From the output tab, researchers can view the Kaplan-Meier estimator curve, see FIG. 14. Referring to FIG. 14, researchers can view the Kaplan-Meier estimator curve in the output tab.

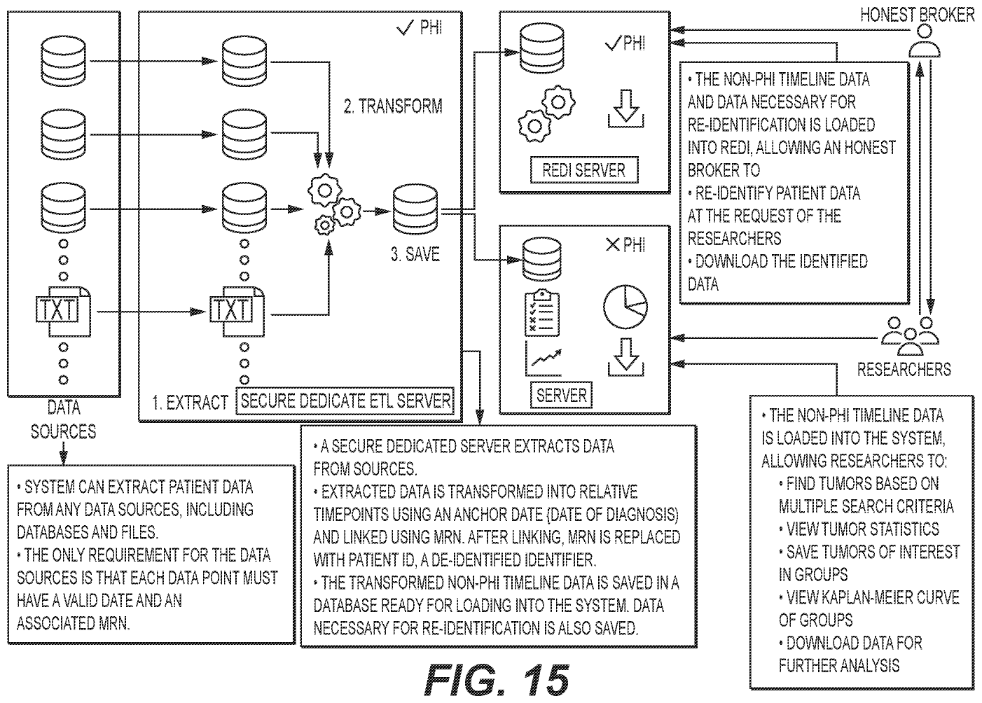

FIG. 15 illustrates an example system architecture and operation according to the system architecture. As described herein, a method of providing clinical and research data of patients from disparate data sources includes extracting patient records from a plurality of disparate data sources native format, wherein each patient record comprises an associated medical record number and a valid date stamp. The extracted patient records are transformed into relative timepoints using an anchor date. The relative time points can be linked using an associated medical record number. The linked records are provided with a patient identification number (e.g., de-identifying the patient records). The linked relative event timepoints are thus non-PHI that can be stored and accessed within a database.

The patient identification number may replace the associated medical record numbers, such that the linked relative event timepoints are de-identified/anonymous/non-patient specific data. Data may be stored for re-identifying the stored data. The relative time points may be loaded into a server and re-identified, e.g. by an honest broker or at the request of an honest broker. The re-identification may be performed at the request of an honest broker. The deidentified data may be downloaded to a platform for access by a subscriber.

The method may also include allowing access to the database for searching based on at least one of the following criteria. The patient records and/or the data source may be at least one of the data type/data sources listed in Table 1. The relative event timepoints may include at least one of the event types listed in Table 2. The database may be searched according to at least one field listed in Table 3. The database storing the linked relative event timepoints may be searchable to find at least one of tumors based on multiple search criteria, tumor statistics, and Kaplan-Meier curve of groups. The data related to the linked relative event timepoints may be downloadable from the database. The deidentified data may be stored in a server separate from identified or re-identified data.

The extracting and transforming may be performed on a secure dedicated ETL Server.

The patient records include PHI and the event timepoints may be non-PHI. The patient records may include a date of diagnosis. The valid time date stamp may include an anchor date. The anchor date may be a date of diagnosis of a patient condition. The patient records may indicate a patient condition. The patient condition may be cancer. The patient condition may be a tumor.

The patient records may include at least one of disease data, intervention data, biospecimen data, diagnostic data, and research data. The disease data may include at least one of diagnostic information, recurrence information, and patient status. The intervention data may include at least one of prescribed drugs, administered drugs, treatment protocols, and surgical procedures. The biospecimen data may include at least one of tumor type/characteristic (solid, liquid, etc.) and tissue microarray. The diagnostic data may include at least one of clinical, genomic, and single analyte information. The research data may include at least one of research sequencing and epidemiological questionnaires and responses.

The event timepoints may include a value of relative days, weeks, months, years, natural log of months and/or percentage of a duration from an anchor date to an end date for each of a plurality of health events for a given patient. The anchor date may be a diagnosis date of a given condition for the given patient. The end date may be a date of death of the given patient.

A system for providing searchable access to de-identified patient data based on a timeline of medical events for the purposes of research, may include a first server comprising a front-end framework, a back-end framework, a search engine, an analytic engine, a database/container; and a second server in electronic communication with the first server and in electronic communication with at least one medical data source, the second server comprising a processor, the processor may store instructions which, when executed by processor, cause the processor to perform operations such as extracting patient records from the at least one medical data source in native format, wherein each patient record comprises an associated medical record number and a valid date stamp; transforming the extracted patient records into relative event timepoints using an anchor date; linking related ones of the relative timepoints using the associated medical record numbers; providing a patient identification number to the linked relative event timepoints; and communicating the linked relative event timepoints as non-protected health information (non-PHI) with an anonymized patient identification number to the first server storing thereon the non-PHI, whereby the non-PHI is anonymized data.

The front-end framework, the back-end framework, the search engine, the analytic engine, and the database/container may be open source including open source code. The second server further may include a proxy server. The proxy server may include open source code. The second server may be in electronic communication with the at least one medical data source via the internet. The second server may be in electronic communication with the at least one medical data source via a dedicate, secure, communication channel.

The system may include access to the second server provided by a secure shell. The shell access may be limited to verified administrators. The second server may be an ETL server. The first server may include a user interface whereby users may search for anonymized patient data in the non-PHI. The system may include a fourth server, whereby an honest broker may re-identify the anonymized patient data via the fourth server. The re-identified patient data may be downloadable via the third server.

The patient records and/or the data source may be at least one of the data type/data sources listed in Table 1. The relative event timepoints may include at least one of the event types listed in Table 2. The database may be searched according to at least one field listed in Table 3. The front-end framework may be implemented in Angular. The back-end framework may be implemented in Django. The search engine, the analytic engine or both may be implemented in Elasticsearch. The database/container may be implemented in MariaDB and/or Nginx. The first server or the second server may include at least one of the technologies listed in Table 4.

A web portal according to principles described herein may include a graphical user interface comprising a display with a workspace; a search interface hosted in the graphical user interface, the search interface providing to a user a graphical representation of search queries to be implemented by a search engine connected to the search interface and in electronic communication with at least one communications portal for sending selected search queries to a database hosted on a server comprising a database of non-protected health information stored as a collection of event timepoints of health events for a given patient.

The event timepoints may include a value of relative days, weeks, months, years, natural log of months and/or percentage of a duration from an anchor date to an end date for each of a plurality of health events for a given patient. The anchor date may be a diagnosis date of a given condition for the given patient. The end date may be the date of death of the given patient. The server may include a front-end framework, a back-end framework, a search engine, an analytic engine, a database/container.

### Advantages & Improvements (Over Existing Methods):

Existing efforts are heavily manual and limited in nature. Investigators will spend a significant amount of time manually piecing together data from various sources to build meaningful research cohorts to facilitate their research ideas. Shared resources are limited in identifying waste via duplicative services on the same research samples. Overall, the current process is slow, tedious and time consuming for Roswell researchers taking away from valuable research activities compared to the utilization of a self-service data discovery technology allowing users to explore and retrieve the needed information on-demand.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1.-55. (canceled)

56. A method of providing clinical and research data of patients from disparate data sources, the method comprising:
extracting patient records from a plurality of disparate data sources in native formats, wherein each patient record comprises an associated medical record number and a valid date stamp;
transforming the extracted patient records into relative event timepoints using an anchor date;
linking related ones of the relative timepoints using the associated medical record numbers;
providing a patient identification number to the linked relative event timepoints; and
storing the linked relative event timepoints as non-protected health information in a database.

57. The method of claim 56, wherein the patient identification number replaces the associated medical record numbers, such that the linked relative event timepoints are de-identified/anonymous/non-patient specific data.

58. The method of claim 57, further comprising storing data for re-identifying.

59. The method of claim 58, further comprising:
loading the relative event timepoints into a server; and
causing the relative event timepoints to be re-identified

60. The method of claim 58, further comprising downloading the identified data to a platform for access by a subscriber.

61. The method of claim 56, wherein the database storing the linked relative event timepoints is searchable to find at least one of:
tumors based on multiple search criteria,
tumor statistics, and
Kaplan-Meier curve of groups.

62. The method of claim 56, wherein the patient records include at least one of disease data, intervention data, biospecimen data, diagnostic data, and research data.

63. The method of claim 62, wherein the disease data includes at least one of diagnostic information, recurrence information, and patient status.

64. The method of claim 62, wherein the intervention data includes at least one of prescribed drugs, administered drugs, treatment protocols, and surgical procedures.

65. The method of any one of claim 62, wherein the biospecimen data includes at least one of tumor type/characteristic and tissue microarray.

66. The method of claim 62, wherein the diagnostic data includes at least one of clinical, genomic, and single analyte information.

67. The method of claim 62, wherein the research data comprises at least one of research sequencing and epidemiological questionnaires and responses.

68. A system for providing searchable access to de-identified patient data based on a timeline of medical events for the purposes of research, the system comprising:
a first server comprising a front-end framework, a back-end framework, a search engine, an analytic engine, and a database/container; and
a second server in electronic communication with the first server and in electronic communication with at least one medical data source, the second server comprising a processor, the processor comprising instructions which, when executed by the processor, cause the processor to perform operations comprising:
extracting patient records from the at least one medical data source in a native format, wherein each patient record comprises an associated medical record number and a valid date stamp;
transforming the extracted patient records into relative event timepoints using an anchor date;
linking related ones of the relative timepoints using the associated medical record numbers;
providing a patient identification number to the linked relative event timepoints; and
communicating the linked relative event timepoints as non-protected health information (non-PHI) with an anonymized patient identification number to the first server storing thereon the non-PHI, wherein the non-PHI is anonymized data.

69. The system of claim 68, wherein the second server further comprises a proxy server.

70. The system of claim 68, wherein the first server further comprises a user interface whereby users may search for anonymized patient data in the non-PHI.

71. The system of claim 68, further comprising a fourth server, whereby an honest broker may re-identify the anonymized patient data via the fourth server.

72. A web portal comprising:
a graphical user interface comprising a display with a workspace;
a search interface hosted in the graphical user interface, the search interface providing to a user a graphical representation of search queries to be implemented by a search engine connected to the search interface and in electronic communication with at least one communications portal for sending selected search queries to a database hosted on a server comprising a database of non-protected health information stored as a collection of event timepoints of health events for a given patient.

73. The web portal of claim 72, wherein the event timepoints include a value of relative days, weeks, months, years, natural log of months and/or percentage of a duration from an anchor date to an end date for each of a plurality of health events for a given patient.

74. The web portal of claim 73, wherein the anchor date is a diagnosis date of a given condition for the given patient and the end date is a date of death of the given patient.

75. The web portal of claim 72, wherein the server comprises a front-end framework, a back-end framework, a search engine, an analytic engine, and a database/container.
